(19) United States
(12) Patent Application Publication
(10) Pub. No.: US 2014/0045205 A1
(43) Pub. Date: Feb. 13, 2014

(54) GLUTAMYL AMINOPEPTIDASE AS A MARKER OF RENAL DAMAGE

(71) Applicants: Antonio Osuna Ortega, Granada (ES); Rosmary Wangensteen Fuentes, Granada (ES); José Félix Vargas Palomares, Granada (ES); Manuel Ramirez Sanchez, Granada (ES)

(72) Inventors: Antonio Osuna Ortega, Granada (ES); Rosmary Wangensteen Fuentes, Granada (ES); José Félix Vargas Palomares, Granada (ES); Manuel Ramirez Sanchez, Granada (ES)

(73) Assignees: UNIVERSIDAD DE GRANADA, Granada (ES); SERVICIO ANDALUZ DE SALUD, Sevilla (ES); UNIVERSIDAD DE JAEN, Jaén (ES)

(21) Appl. No.: 13/988,464

(22) PCT Filed: Nov. 10, 2011
(86) PCT No.: PCT/ES11/00332

§ 371 Date: Aug. 9, 2013

(30) Foreign Application Priority Data
Nov. 18, 2010 (ES) P201031699

(51) Int. Cl. C12Q 1/37

(52) U.S. Cl. CPC C12Q 1/37
USPC 435/24

(57) ABSTRACT

The present invention relates to a method and a kit for the diagnosis and/or prognosis of kidney injury comprising analysing a sample obtained from a patient and determining the activity of at least one aminopeptidase selected from aspartyl aminopeptidase, glutamyl aminopeptidase, alanyl aminopeptidase and leucyl-cystinyl aminopeptidase.

### FIELD OF THE INVENTION

The present invention is generally classified in the field of biomedicine and in particular relates to a method for the diagnosis and/or prognosis of acute kidney injury.

### PRIOR ART

The Acute Kidney Injury Network (AKIN) describes acute kidney injury as the “functional or structural abnormalities or markers of kidney damage including abnormalities in blood, urine, or tissue tests or imaging studies present for less than three months” (Vaidya V S, Ferguson M A, Bonventre J V. Biomarkers of acute kidney injury. Annu Rev Pharmacol Toxicol 2008; 48: 463-493).

Normally, acute kidney injury is associated with the retention of creatinine, urea, and other metabolic waste products that are normally excreted by the kidney.

Although in more severe cases it may result in oliguria or even anuria, urine volume may be normal or even increased (Mehta R L, Kellum J A, Shah S V, Molitoris B A, Ronco C, et al. Acute Kidney Injury Network: report of an initiative to improve outcome in acute kidney injury. Crit Care 2007; 11: R31).

Acute kidney injury has various etiologies whether due to a decrease in renal or intrarenal perfusion, due to a toxic aggression or obstruction of the kidney tubule, due to tubulointerstitial inflammation and edema or due to a reduction in the glomerule filtration capacity (Chertow G M, Lee J, Kuperman G J, Burdick E, Horsky J, et al. Guided medication dosing for inpatients with renal insufficiency. JAMA 2001; 286: 2839-2844). It is a complex pathology with a mortality rate (50-70%) that has remained fairly invariable in the last 50 years (Uchino S, Kellum J A, Bellomo R, Doig G S, Morimatsu H, et al. Acute renal failure in critically ill patients: a multinational, multicenter study. JAMA 2005; 294: 813-818.).

Various physiopathological mechanisms contribute to the development and progression of acute kidney injury: increase in renal vasoconstriction, tubular dysfunction and cell death due to apoptosis or necrosis, cell desquamation, abnormalities in the transport of ions that lead to an imbalance in the tubuloglomerular balance, and local production of inflammation mediators that cause interstitial inflammation and vascular congestion (Bonventre J V, Weinberg J M. Recent advances in the pathophysiology of ischemic acute renal failure. J Am Soc Nephrol 2003; 14: 2199-2210; Schrier R W, Wang W, Poole B, Mitra A.

Acute renal failure: definitions, diagnosis, pathogenesis, and therapy. J Clin Invest 2004; 114: 5-14.).

The most widely used markers in clinical practice for the detection of kidney injury are urea and serum creatinine which have limitations in terms of sensitivity and specificity. Furthermore, the kidney injury must affect more than 50% of the renal function so that a rise occurs in the urea and serum creatinine (Devarajan, P. Emerging biomarkers of acute kidney injury. Acute kidney Injury 2007; 156: 203-212; Bonventre J V, Vaidya V S, Schmouder R, Feig P, Dieterle F. Next-generation biomarkers for detecting kidney toxicity. Nat Biotechnol 2010; 28: 436-440.)

The early detection of kidney injury currently continues to be a challenge, both in clinical practice and in pharmacological toxicity assays, and biomarkers are needed that allow its early diagnosis, as well as its evolution, with urinary biomarkers being the most promising in this regard.

An ideal urinary marker must be highly organ-specific, it must allow the recognition of the etiology of kidney injury (hypoxia, toxins, sepsis, etc.), it must correlate with the histological findings in renal biopsies, it must detect the early damage and identify pathological changes in the different tubular segments, it must correlate with the degree of tubular damage and have high sensitivity for the early detection both of minimal changes and of the appearance of a more severe damage (the marker must be detectable throughout the course of the kidney injury with threshold values defined to monitor the progression and regression of kidney injury; to date, there is no suitable method to differentiate benign, mild, moderate or severe renal dysfunction), its analysis must not be invasive and, finally the laboratory analyses must be simple and quick to carry out, safe, reproducible, cheap and that allow the serial determination of a large number of samples (Lisowska-Myjak B. Serum and urinary biomarker of acute kidney injury. Blood purif 2010; 29: 357-365.).

The characterization of these urinary markers must serve to elucidate if they are useful tools in the early diagnosis, if they identify the kidney injury mechanism, and/or if they indicate the site and severity of the damage, helping to monitor response to the treatment.

The markers of acute kidney injury can be classified as (Lisowska-Myjak B. Serum and urinary biomarker of acute kidney injury. Blood purif 2010; 29: 357-365.):
1) Enzymes released from tubular cells that are damaged, dysfunctional or with necrosis/apoptosis: alkaline phosphatase, gamma-glutamyl transpeptidase, alanine aminopeptidase, glutathione transferase isoenzymes, N-acetyl-β-D-glucosanninidase (NAG).
2) Low molecular weight proteins (<40 kDa) (α-microglobulin, β₂-microglobulin, retinol binding protein (RBP), cystatin C), the presence of which in urine reflects a decrease in reabsorption by the proximal tubule cells,
3) Proteins specifically produced in the kidney during the course of kidney injury: cysteine-rich protein 61, neutrophil gelatinase associated lipocalin (NGAL), kidney injury molecule (KIM-1), cytokines and chemokines (Gro-α, IL-18), and
4) Structural and functional proteins of the renal tubules: F-actin, sodium/hydrogen exchanger (NHE-3).

Different markers of acute kidney injury have been determined, Nguyen Mai T et al. “Biomarkers for the early detection of acute kidney injury”. Pediatric Nephrology. Dec. 2008. Vol. 23. No. 12, pages 2151-2157. describes NGAL, II-18, KIM-1 and Cystatin C as markers of acute kidney injury.

Ferguson et al. “Biomarkers of nephrotoxic acute kidney injury”. TOXICOLOGY. Apr. 1, 2008. Vol. 245, No. 3, pages 182-193. cites Alanyl aminopeptidase as one of the proteins with enzymatic activity as marker of acute kidney injury.

Trof Ronald J. et al. “Biomarkers of acute renal injury and renal failure”. Shock. Sep. 2006. Vol. 26. No.. 3, pages 245 - 253. relates to serological and urinary markers to detect acute kidney dysfunction and injury, within the urinary markers it cites Ala-(Leu-Gly) aminopeptidase.

Nomura M. et al. “Possible involvement of aminopeptidase A in hypertension and renal damage in Dahl salt-sensitive rats”. American Journal of Hypertension. Jan. 4, 2005. Vol. 18. No. 4, pages 538-543. describes that glutamyl aminopeptidase is decreased in the kidney tissue in DS and in DR rats and this decrease could be involved in the evolution of hypertension and kidney injury.

Scherberich J. E. et al. “Immunological and ultrastructural analysis of loss of tubular membrane-bound enzymes in patients with renal damage”. Clinica chimica acta. 15 Dec. 1989. Vol.185, No. 3, pages 271 - 282. describes aminopeptidase M, also called (Ala-(Gly-leu)-aminopeptidase or Alanyl-aminopeptidase, as membrane disruption marker in the proximal tube.

In Solis-Herruzo J. A. et al “Urinary excretion of enzymes in cirrhotics with renal failure”. Journal of Hepatology. 1986. Vol. 3, No.. 1, pages 123-130, the authors confirm that the measurement of urinary enzymes, among which we have leucine aminopeptidase, has a diagnostic value of kidney injury in cirrhotic patients.

Serrera Contreras J. L. et al. “Enzinnologia del clan° renal agudo”. Rev Clin Esp. 1972 Vol. 30; No. 127(4), pages 855-862. describes that leucine aminopeptidase has a diagnostic value in acute kidney injury.

All the aforementioned justifies the need to identity and validate new, more precise kidney injury biomarkers, whose determination is also fast, simple and without the need to biopsy the patient.

### Explanation of the Invention

The present invention provides a non-invasive method to differentiate between benign, mild, moderate or severe kidney dysfunction, which can be easily used in daily clinical practice, by a simple, fast and economical test that allows the serial determination of a high sample volume.

Thus, in a first aspect, the present invention relates to the use of at least one aminopeptidase selected from aspartyl aminopeptidase, glutamyl aminopeptidase, alanyl aminopeptidase and leucyl-cystinyl aminopeptidase as markers of diagnosis and/or prognosis of kidney injury.

In a second aspect, the present invention relates to a method for obtaining useful data in the diagnosis and/or prognosis of kidney injury comprising determining in an isolated sample the activity of at least one aminopeptidase selected from the group comprising: aspartyl aminopeptidase, glutamyl aminopeptidase, alanyl aminopeptidase and leucyl-cystinyl aminopeptidase, or any combinations thereof. In a preferred embodiment, the activity of the aminopeptidases aspartyl aminopeptidase, glutamyl aminopeptidase, alanyl aminopeptidase and leucyl-cystinyl aminopeptidase is determined simultaneously. In a particular embodiment, the activity of the aminopeptidase(s) is determined by fluorometric analysis.

In a third aspect, the present invention relates to a method for the diagnosis and/or prognosis of kidney injury comprising analysing a sample obtained from a patient, determining the activity of at least one aminopeptidase selected from the group comprising: aspartyl aminopeptidase, glutamyl aminopeptidase, alanyl aminopeptidase and leucyl-cystinyl aminopeptidase, or any combinations thereof, and comparing said activity with a control value, where the alteration of said activity is indicative of kidney injury. In a preferred embodiment, the activity of the aminopeptidases aspartyl aminopeptidase, glutamyl aminopeptidase, alanyl aminopeptidase and leucyl-cystinyl aminopeptidase is determined simultaneously.

In the present invention, by kidney injury we refer, without limiting ourselves, to any functional or structural abnormality of various etiologies whether due to a decrease in renal or intrarenal perfusion, due to a toxic aggression or obstruction of the kidney tubule, due to tubulointerstitial inflammation and edema or due to a reduction in the glomerule filtration capacity.

In a more preferred embodiment of the present invention, the kidney injury that can be diagnosed and/or prognosed is an acute kidney injury.

In the present invention, by acute kidney injury we refer to a kidney injury where the functional or structural abnormality that produces it is present during at least three consecutive months.

In another more preferred embodiment of this aspect of the invention, the sample analyzed is a urine sample.

In another more preferred embodiment the aminopeptidase activity is determined by fluorometric analysis.

In another preferred embodiment, the fluorometric analysis is carried out by the use of a substrate selected from glutamyl-β-naphthylamide, alanyl-β-naphthylamide, aspartyl-β-naphthylamide and/or cystinyl-β-naphthylamide.

In another preferred embodiment, the increase in the activity of aspartyl aminopeptidase, glutamyl aminopeptidase, alanyl aminopeptidase and/or leucyl-cystinyl aminopeptidase with respect to control value is indicative of kidney injury.

In another preferred embodiment, the increase in the activity of glutamyl aminopeptidase and/or alanyl aminopeptidase with respect to control value is indicative of early kidney injury.

In the present invention, by early kidney injury we refer to kidney injury in the first stages of its evolution which is accompanied with minimal changes that are difficult to detect using typical analytical methods, and which is translated into a benign or mild kidney dysfunction.

In another preferred embodiment, when the patient has been diagnosed with early kidney injury according to the method of the present invention, the decrease in the activity of glutamyl aminopeptidase and/or alanyl aminopeptidase is indicative of recovery of renal function.

Therefore, the present invention in a particular embodiment relates to glutamyl aminopeptidase and/or alanyl aminopeptidase as markers for the early diagnosis of kidney injury.

In another preferred embodiment, the increase in the activity of aspartyl aminopeptidase and/or leucyl-cystinyl aminopeptidase is indicative of serious renal dysfunction.

In the present invention by serious renal dysfunction we refer to kidney injury in an advanced phase of its evolution which is accompanied with hematological, urinary and histological alterations and entails a moderate or severe decrease of renal function.

Therefore, a preferred embodiment relates to aspartyl aminopeptidase and/or leucyl-cystinyl aminopeptidase as markers for the prognosis of kidney injury.

In another preferred embodiment, the patient suffers from another disease of different etiology to kidney injury, in another preferred embodiment the patient suffers a disease selected from hyperthyroidism, hypertension, diabetes, transplant, secondary effects of drugs that cause kidney injury.

In a fourth aspect, the present invention relates to a kit or device, hereinafter kit or device of the invention, for the diagnosis and/or prognosis of kidney injury according to the method described above, comprising the necessary reagents to determine the activity of aspartyl aminopeptidase, glutamyl aminopeptidase, alanyl aminopeptidase and/or leucyl-cystinyl aminopeptidase.

In a preferred embodiment, the kit comprises the substrates necessary to determine the activity of aspartyl aminopeptidase, glutamyl aminopeptidase, alanyl am inopeptidase and/or leucyl-cystinyl aminopeptidase using a fluorometric method. In a preferred embodiment, the kit comprises glutamyl-β-naphthylamide, alanyl-β-naphthylamide, aspartyl-β-naphthylamide and/or cystinyl-β-naphthylamide.

In a fifth aspect, the present invention relates to the use of the kit described above for the diagnosis and prognosis of kidney injury.

### BRIEF DESCRIPTION OF THE DRAWINGS

FIG. 1 shows a) proteinuria, b) GluAp activity, c) AlaAp activity, d) AspAp activity and e) CysAp activity, in control rats, rats with 8% salt in the diet (Salt), hypothyroid rats treated with 300 mg/l methimazole in drinking water (Methimazole), rats with 8% salt+methimazole (Methimazole+Salt), hyperthyroid rats treated with thyroxine (200 μg.kg⁻¹.day⁻¹) (T4) and rats with 8% salt+thyroxine (T4+Salt). 1 p<0.05, 2 p<0.01, 3 p<0.001 against its respective Control group or Salt; 4 p<0.05, 5 p<0.01, 6 p<0.001 against its respective group treated with Methimazole or with T4. n=6 in each group.

FIG. 2 shows a) Proteinuria, b) GluAp activity, c) AlaAp activity, d) AspAp activity and e) CysAp activity in rats treated with cisplatin at doses of 3.5 and 7 mg/kg. * p<0.05, ** p<0.01 versus baseline. n=5 in each group.

FIG. 3 shows a) GluAp, b) AlaAp, c) AspAp and d) CysAp activity in healthy controls and patients with kidney injury. * p<0.05, ** p<0.01 versus control. Control (n=5). Patients (n=6).

FIG. 4 shows a) GluAp activity, b) AlaAp, c) AspAp and d) CysAp 2, 4 and 5 days after kidney transplant in two patients.

FIG. 5 shows the evolution of a) GluAp activity, b) AlaAp activity, c) AspAp activity and d) CysAp activity 2, 4 and 5 days after kidney transplant. * p<0.05, ** p<0.01 versus control. Control (n=5). Transplanted (n=4).

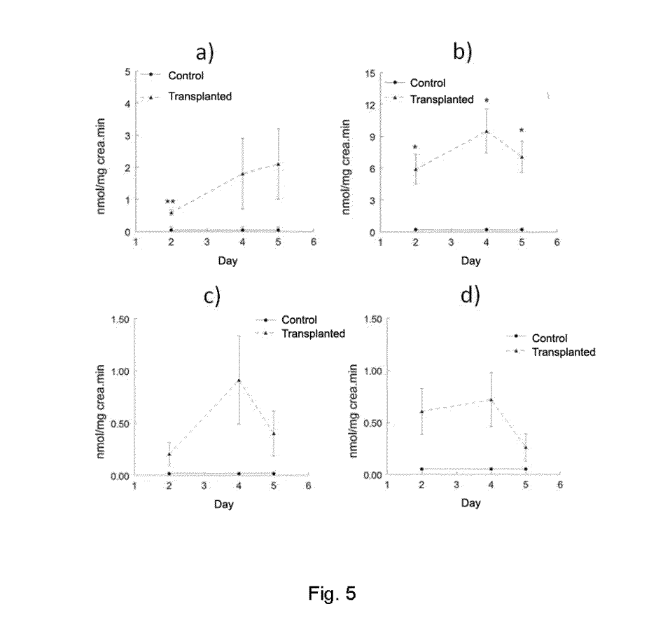

### DETAILED EXPLANATION OF EMBODIMENTS

### Example 1

### Analytical Determination of GluAp, AlaAp, AspAp and CysAp Activities in Animal Models

On the one hand, 42 male Wistar rats of 250 g in weight from the animal house of the University of Valladolid were distributed in 6 groups (n=7) with the following treatments: Control, Salt (8% NaCl in the diet), Hypothyroid (methimazole, 300 mg/l in drinking water), Hypothyroid+Salt, Hyperthyroid (daily s.c. injection of thyroxine (T4) 200 μg.kg⁻¹.day⁻¹), and Hyperthyroid+Salt.

All treatments were maintained during 6 weeks.

At the end of the experiment, and after a 2-day period of adaptation to the metabolic cages, each animal was introduced in a metabolic cage during 24 hours. The dieresis was determined, and the urine was centrifuged for 10 min at 1,000 g, aliquoted and frozen at −80° C. until its use.

In another experiment, 15 male Wistar rats were used of 250 g in weight provided by Harlan Laboratories, which were distributed in 3 groups (n=5): Control, Cisplatin 3.5 (a single s.c. injection on the first day of the experiment of cisplatin at doses of 3.5 mg/kg in weight), and Cisplatin 7 (s.c. injection of 7.5 mg/kg cisplatin).

After a period of 2 days of adaptation to the metabolic cages, a baseline sample of diuresis was taken before the treatment, and then the urine was collected from the treated groups on days 2, 3, 7 10 and 14, also taking two samples from the control rats, at the start and end of the experiment.

The GluAp, AlaAp, AspAp and CysAp activities were determined in duplicate by a fluorometric analysis using glutamyl-, alanyl-, aspartyl- and cystinyl-β-naphthylamide as substrates, respectively.

Hence, 20 μl of urine were incubated during 60 minutes at 37° C. with 90 μl of its corresponding substrate solution:
2.72 mg/dl glutamyl-β-naphthylamide, 10 mg/dl BSA, 10 mg/dl DTT and 555 mg/dl CaCl₂ in 50 mM Tris-HCl pH 7.4,
2.14 mg/dl alanyl-β-naphthylamide, 10 mg/dl BSA, 10 mg/dl DTT in 50 mM Tris-HCl pH 7.4,
2.58 mg/dl aspartyl-β-naphthylamide, 10 mg/dl BSA, 39.4 mg/dl MnCl₂ in 50 mM Tris-HCl pH 7.4,
5.63 mg/dl cystinyl-β-naphthylamide, 10 mg/dl BSA; 10 mg/dl DTT in 50 mM Tris-HCl pH 6.

The substrates had been previously dissolved in 1 ml of DMSO and stored at -20° C.

The quantity of β-naphthylamine released as a result of the aminopeptidase activities was measured fluorometrically at an emission wavelength of 412 nm with excitation at 345 nm, taking a measurement of the fluorescence after 60 minutes, after stopping the reaction with 90 μl of 0.1 M acetate buffer.

A blank was performed in duplicate for each sample. To do this, a procedure identical to the above was followed, but using an incubation solution that did not contain the corresponding substrate. The fluorescence difference between both measurements was quantified using a standard curve of β-naphthylamine dissolved in water at the concentrations of 12.5, 25, 50, 100 and 200 nmol/ml 90 μl of the incubation solution corresponding to each enzyme without the substrate were added to 20 μl of this solution, they were incubated during 60 minutes at 37° C., and 90 μl of 0.1 M acetate buffer were added. It was verified that the presence of the substrate in the incubation solutions in no way modified the fluorescence of the standard. All measurements were made in duplicate, and the linearity of the fluorescence obtained in the curve range was verified (0-200 nmol/ml).

Both for the determinations of the samples and for the blanks, reaction blanks were performed containing 20 μl of distilled water, 90 μl of incubation solution with or without substrate, respectively, and 90 μl of sodium acetate.

The specific activity of each aminopeptidase was expressed as nanomoles of hydrolyzed substrate per minute and per mg of creatinine. The linearity of the fluorescence obtained was verified with respect to hydrolysis time and creatinine content in a same sample.

Urinary creatinine was determined using Jaffe's kinetic method, based on the reaction of creatinine with sodium picrate.

Proteinuria was determined using a commercial kit from Bio-Rad (DC Protein Assay kit) based on Lowry's method.

After performing a variance study, a t-test was performed for unpaired data assuming equal variances. In the cases where the samples did not fit normality, Fischer's modification was performed for unequal variances. It was taken as significant difference when p<0.05 versus group control.

In the case of the rats subjected to treatment with cisplatin, a t-test was performed for paired data with respect to the baseline sample of each animal. It was taken as significant difference when p<0.05 versus baseline.

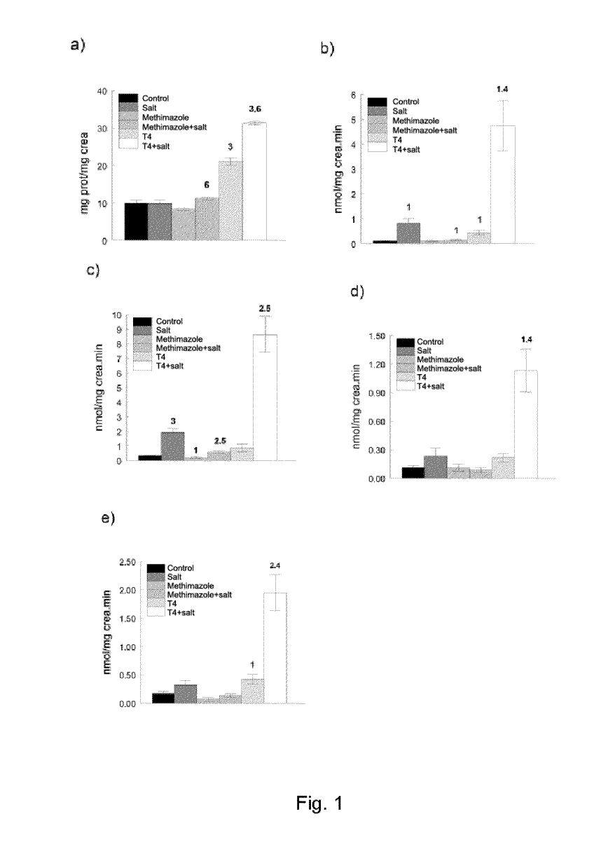

The results showed that the rats treated with 8% Salt had a significant increase in two of the four enzymes analyzed: GluAp and AlaAp. The increase in these tubular enzymes was not accompanied with proteinuria (FIG. 1) or an increase in plasma creatinine.

In hypothyroidism no modification was observed in the proteinuria or in the aminopeptidase activity, except for AlaAp which was significantly decreased. The administration of methimazole to the rats treated with salt (Hypothyroid+salt) also caused a decrease, both of GluAp and AlaAp in comparison with the rats treated with salt (FIG. 1).

At the end of the treatment, the rats treated with T4 had a significant increase in GluAp urinary activity, as well as the proteinuria level excreted per mg of creatinine (FIG. 1). The simultaneous administration of salt to the hyperthyroid rats caused a great increase in the 4 enzymes and proteinuria. GluAp and AlaAp rose more than tenfold, and CysAp and AspAp around fivefold over the value of activity of the hyperthyroid rats.

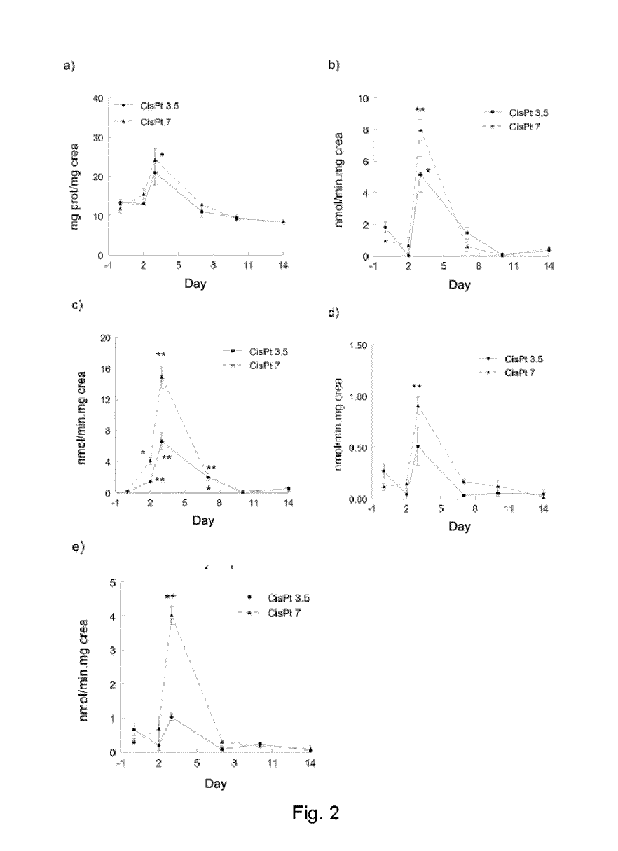

In the case of the rats treated with cisplatin at doses of 3.5 mg/kg, a slight increase was observed 3 days after treatment in proteinuria levels (1.5 times above the baseline level), which did not become statistically significant, whilst, however, the GluAp and AlaAp activities rose 3 and 60 times, respectively (FIG. 2)

In the animals treated with the dose of 7 mg/kg, on the third day of treatment, the proteinuria doubled, whilst the GluAp and the AlaAp rose 8 and 70 times, respectively.

A rise in CysAp and AspAp activity was only observed in the group treated with cisplatin at doses of 7 mg/kg.

### Example 2

### Analytical Determination of GluAp, AlaAp, AspAp and CysAp Activities in Patients

Urine samples were collected from patients with kidney injury (n=6) and from healthy controls (n=5), and the urine was centrifuged for 10 min at 1,000 g, aliquoted and frozen at −80° C.

In two of these patients, subjected to kidney transplant, urine samples were collected on 2, 4 and 5 days after the transplant.

The GluAp, AlaAp, AspAp and CysAp activities were determined as explained in example 1.

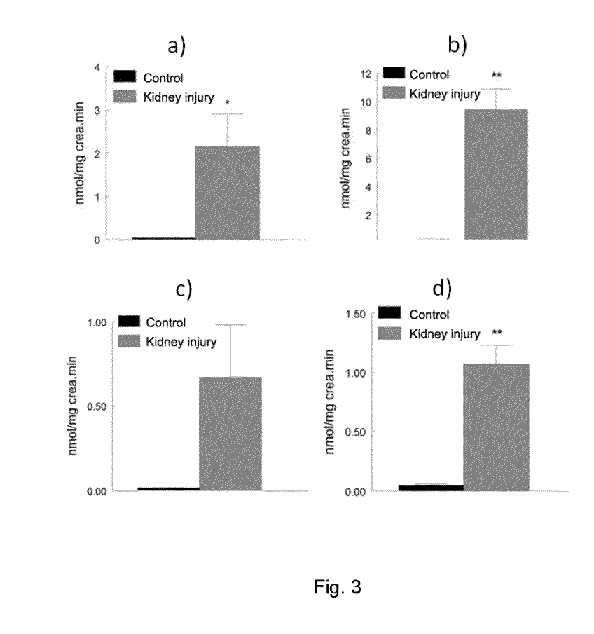

In the 6 cases analyzed a significant increase was observed of GluAp, AlaAp and CysAp, with AlaAp being the one that reached greater activity values (FIG. 3). AspAp was increasing in five of the six patients analyzed.

The control level for GluAp was 0.038±0.009 nmol/mg crea.min, for AlaAp it was 0.197±0.047 nmol/mg crea.min, for AspAp it was 0.016±0.003 nmol/mg crea.min and for CysAp it was 0.051±0.010 nmol/mg crea.min.

The two patients wherein the enzymatic activity was determined in the days after the kidney transplant had a great increase in these enzymes.

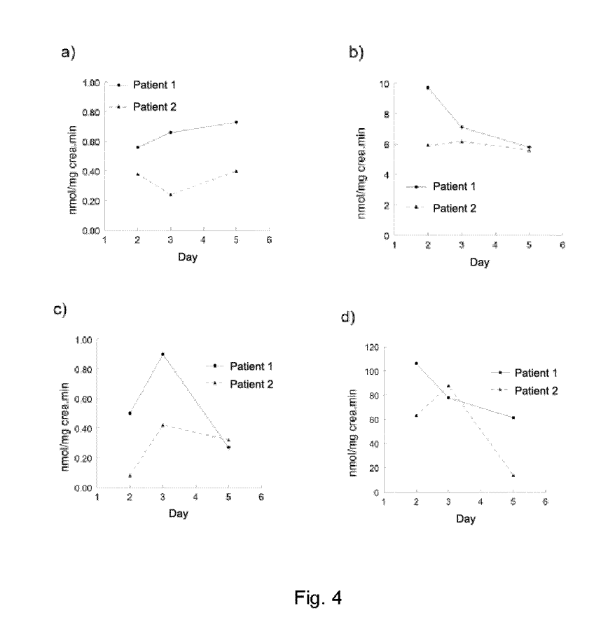

In the case of patient 1, 5 days after the transplant the enzymes began to decrease, three of them being below the level they had at 2 days. The blood creatinine remained high, but a certain improvement in creatinine clearance was observed, as well as proteinuria decrease (FIG. 4, table 1).

TABLE 1
Values of blood creatinine, creatinine clearance and proteinuria in
the days after the kidney transplant of the two patients studied.

| | Day 2 | Day 4 | Day 5 |
|---|---|---|---|
| Patient 1 | | | |
| Blood creatinine (mg/dl) | 7.70 | 9.60 | 8.76 |
| Crea Clearance (ml/min) | 6.20 | 8.24 | 18.24 |
| Proteinuria (mg/mg crea) | 18.6 | 10.9 | 7.62 |
| Patient 2 | | | |
| Blood creatinine (mg/dl) | 12.2 | 11.4 | 9.2 |
| Crea Clearance (ml/min) | 5.09 | 8.14 | 10.8 |
| Proteinuria (mg/mg crea) | 12.1 | 9.75 | 10.3 |

In the case of patient 2, only a clear decrease in CysAp was observed on day 5 after the transplant, with the other three enzymes remaining high. However, a progressive decrease was observed in the blood creatinine, and a very slight increase in the clearance. The proteinuria maintained at high levels (FIG. 4, table 1).

The fact that in the animals treated with cisplatin there was a large rise in GluAp and AlaAp 3 days after the injection, with a slight increase in the proteinuria, and in the rats treated with 8% salt there was also an increase in these two enzymes without there being an increase in the excretion of protein, urea or plasma creatinine, indicated that these aminopeptidases, in particular GluAp and AlaAp, were of great use in the early detection of kidney injury.

AspAp and CysAp activities only rose in the event of serious renal dysfunction, rising much more in the hyperthyroid rats treated with salt than in the hyperthyroid rats that followed a normal diet, in the rats treated with cisplatin at doses of 7 mg/kg, and with very high levels appearing in patients with studied kidney injury, although in one case the AspAp level was within normality.

Furthermore, in the rats treat with salt that were administered methimazole (Hypothyroid+salt) a decrease occurred in GluAp and AlaAp with respect to the rats treated with salt, due to the protective renal effect of methimazole. Additionally, in the transplanted patients, we observe how these enzymes decreased as renal function recovered.

The results shown in the examples of the present invention indicated that these enzymes were indicators of the progression and regression of kidney injury, differentiating between mild or severe damages in a multitude of pathological situations affecting the kidneys: glomerulonephritis, chronic nephropathies, renal insufficiency, treatments with antitumor drugs, diabetic patients, hypertensive patients, etc.

1. (canceled)

2. A method for obtaining useful data in the diagnosis and/or prognosis of kidney injury comprising determining in an isolated sample the glutamyl aminopeptidase activity.

3. The method according to claim 2, where the aminopeptidase activity is determined by fluorometric analysis.

4. A method for the diagnosis and/or prognosis of kidney injury comprising determining in an isolated sample the glutamyl aminopeptidase activity and comparing said activity with a control value, where the alteration of said activity is indicative of kidney injury.

5. The method according to claim 4, where the kidney injury is an acute kidney injury.

6. The method according to claim 4, where the sample is urine.

7. The method according to claim 4, where the aminopeptidase activity is determined by fluorometric analysis.

8. The method according to claim 4, where the substrate used is glutamyl-β-naphthylamide.

9. The method according to claim 4, where the increase in the glutamyl aminopeptidase activity, with respect to control value is indicative of kidney injury.

10. The method according to claim 4, where the increase in the glutamyl aminopeptidase activity with respect to control value is indicative of early kidney injury.

11. The method according to claim 4, where the patient suffers a disease selected from the group consisting of hyperthyroidism, hypertension, diabetes, transplant, and secondary effects of drugs that cause kidney injury.

12. A kit for the diagnosis and/or prognosis of kidney injury according to the method of claim 4, comprising the reagents necessary to determine the glutamyl aminopeptidase activity.

13. The kit according to claim 12, comprising glutamyl-β-naphthylamide.

14. (canceled)
